(19) United States
(12) Patent Application Publication
(10) Pub. No.: US 2009/0069743 A1
(43) Pub. Date: Mar. 12, 2009

(54) INFUSION THERAPY SENSOR SYSTEM

(75) Inventors: SIVARAMAKRISHNAN KRISHNAMOORTHY, Lake Zurich, IL (US); SANJUN NIU, Lake Villa, IL (US); BIRENDRA K. LAL, Palatine, IL (US); TUAN BUI, Green Oaks, IL (US); RANDOLPH R. MEINZER, Spring Grove, IL (US)

Correspondence Address: BAXTER HEALTHCARE CORPORATION, ONE BAXTER PARKWAY, DF2-2E, DEERFIELD, IL, 60015, US

(73) Assignees: BAXTER INTERNATIONAL INC., DEERFIELD, IL (US); BAXTER HEALTHCARE S.A., ZURICH (CH)

(21) Appl. No.: 12/208,367

(22) Filed: Sep. 11, 2008

Related U.S. Application Data
(60) Provisional application No. 60/971,449, filed on Sep. 11, 2007.

(51) Int. Cl. A61M 5/168; A61B 5/1455

(52) U.S. Cl. 604/66; 600/310

(57) ABSTRACT

A sensor system for use with an infusion system may include at least one sensor disposed within a catheter, the at least one sensor comprising at least one of an optical sensor, an electrical sensor or a chemical/biochemical sensor. The sensor system may instead include a sample cell that is in fluid communication with the infusion system, which sample cell may be used with an analyzer to determine a patient's condition. The sensor system may be integrated with a control system for an infusion pump to control operation of the pump.

### BACKGROUND

This patent relates to a system for sensing a patient's condition in association with an infusion therapy system. In particular, this patent relates to sensing, diagnosis, theranosis, prognosis and/or analysis of a patient's condition based on a sample of bodily fluid and, potentially, analyte obtained within a patient or from an infusion line.

One pressing problem encountered in health-care situations is the need for real-time information regarding a patient's condition, for example, to change or alter a therapy. A related problem is the need to detect if a particular event has occurred so that timely intervention by the health care provider may be accomplished.

To acquire such clinically useful information, sensors and other hardware systems have been employed. Typically, the sensors and other systems have used different technologies to sense different parameters, such as blood pressure, blood gas, blood chemistry, glucose, drugs, etc. Based on the technology used, sensors may be classified as electrical, optical or biochemical sensors.

The sensors may be disposed in the body or located outside the body. In-vivo sensors are disposed inside the body of the patient, such as tip of infusion catheter, while in-vitro sensors are located outside the body such as in the infusion line or offline. Both sensors pose variety of challenges in their design and development.

For example, sensitivity of an in-vivo optical sensor depends on the intensity of the light that is collected at the receiver after it has been transmitted or fluoresced or scattered through the whole blood. Since blood absorbs light very well, the intensity of incident light may be increased; however, too high an intensity may damage the blood cells and impact the accuracy of the sensed parameter. A method of alleviating this absorption problem is to decrease the “optical distance” defined as the distance light has to travel through the blood between the emitter and collector, but this can cause issues as well. Further, in the case of electrical and biochemical sensors, use of reagents pose significant challenges for the reasons of biocompatibility, toxicity and reuse.

On the other hand, in-vitro sampling typically does not occur in real time. Once an in-vitro sample is drawn, even if the laboratory is on-site and laboratory personnel treat the sample without delay, it may take anywhere between 20-30 minutes to a day to complete the analysis and present a result. Further delays may result because of sampling protocol and laboratory procedures. Such delays hinder the ability of the healthcare practitioners to make changes to on-going therapies.

As a further complication, no one known sensor or sensor system can sense all required information. As a consequence, it becomes necessary to use a combination of sensors to obtain all of the required information. The individual sensors within the combination of sensors typically must be placed at different locations on or in the patient. For example, a sensor to measure arterial blood pressure is placed in an arterial line, while a sensor to measure venous blood pressure is placed in a venous line. Consequently, the patient conventionally has to be accessed at multiple sites.

Having multiple sensors and multiple access sites can create additional problems. For one thing, the use of multiple sensors can change clinician workflow, and require a higher level of skill on the part of the healthcare practitioner to operate the sensors. Additionally, the use of multiple access sites may increase infection risk and patient discomfort.

At the same time, it is known that one prevalent way of providing therapy to a patient is to employ infusion therapy, where fluids are administered intravenously with the composition of the fluids varying depending on the need of the patient. In most infusion therapy, a catheter is inserted into the venous system of a patient. The catheter is in fluid communication with the contents of one or more intravenous (IV) containers through the use of an administration set. An infusion pump may also be employed for tight control of the rate of infusion.

What is needed is a device that would provide a real-time sensing of the patient's condition. An additional need is a system that may be used to rapidly test the body fluids of a patient, including blood, saliva, and urine, and potentially the infusate from an infusion therapy system, including IV solution such as saline, medication, and blood. A further need is to perform this sensing while minimizing the patient's discomfort and infection risk and the healthcare practitioner's required skill level.

As set forth in more detail below, the present disclosure sets forth an improved assembly embodying advantageous alternatives to the conventional devices and approaches discussed above.

### SUMMARY

According to an aspect of the present disclosure, an integrated sensor system for providing information to a control system is provided. The sensor system includes a catheter configured for communication with the control system, the catheter forming at least one lumen. The sensor system also includes at least one sensor disposed within the catheter, the at least one sensor comprising at least one of an optical sensor, an electrical sensor or a chemical/biochemical sensor.

According to another aspect of the present disclosure, an integrated sensor system includes an infusion pump, a control system operably connected to the infusion pump, and a multi-lumen catheter in fluid communication with the infusion pump. The sensor system also includes at least one sensor disposed within the catheter, the at least one sensor comprising at least one of an optical sensor, an electrical sensor or a chemical/biochemical sensor. The sensor is operably connected to the control system, and the control system is configured to receive and process input signals from the at least one sensor and to provide an output useful for a real-time diagnosis.

According to still another aspect of the present disclosure, a sensor system includes a sample cell including opposing walls spaced from each other to define a test region therebetween and an inlet in fluid communication with the test region, and an analyzer. The analyzer includes a housing comprising a holder in which at least the test region of the sample cell is received, and a light emitter and a light receptor, the light emitter and the light receptor disposed about the holder adjacent to the test region. The analyzer also includes a processor operatively coupled to the light receptor to receive a sensor signal therefrom, the processor programmed to determine a physical condition of a patient according to the sensor signal, and a signaling device operatively coupled to the processor to receive a processor signal therefrom, the signaling device providing an indication associated with the physical condition of the patient according to the processor signal.

According to yet another aspect of the present disclosure, a sensor system disposable includes an administration set connector, a catheter hub connector; and a sensor cell including opposing walls spaced from each other to define a test region therebetween. The sample cell is connected at a first end to the administration set connector and at a second end to the catheter hub connector.

Additional aspects of the disclosure are defined by the claims of this patent.

### BRIEF DESCRIPTION OF THE FIGURES

It is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 is a schematic view of an integrated infusion pump and intravenous sensor system;

FIG. 2 is a closer view of an embodiment of the connections between the pump and the system;

FIG. 3 is a closer view of a controller for the embodiment of FIGS. 1-2;

FIG. 4 is a first embodiment of a catheter system;

FIG. 5 is a second embodiment of a catheter system;

FIGS. 6A and 6B are side cross-sectional views of the distal end of the catheter system of FIG. 4;

FIGS. 7A-7H are cross sectional and side views of the embodiment of FIG. 6A and 6B;

FIG. 8 is a cross sectional view of the embodiment of FIG. 5;

FIG. 9 is a side view of another catheter embodiment;

FIG. 10 is another schematic view of an integrated infusion pump and sensor system;

FIG. 11 is a plan view of an embodiment of an extension set system for use with an infusion pump;

FIG. 12 is a cross-sectional view of the extension set system of FIG. 11 taken at line 12-12;

FIG. 13 is a schematic view of an analyzer with the two sections of the analyzer spaced apart to view the internals of the analyzer;

FIG. 14 is a cross-sectional view of the analyzer of FIG. 13 taken at line 14-14;

FIG. 15 is plan view of a sensor system including the extension set system of FIG. 11 and the analyzer of FIG. 13, with the extension set system being inserted into a holder of the analyzer;

FIG. 16 is a plan view of the sensor system of FIG. 15 with the extension set system received in the holder of the analyzer;

FIG. 17 is a plan view of an embodiment of an extension set system having an integrated pump and valve;

FIG. 18 is a partial, perspective view of an embodiment of an extension set system with a frame supporting a sensor cell, pump, and valve;

FIG. 19 is a cross-sectional view of the extension set system of FIG. 18 taken at line 19-19;

FIG. 20 is a perspective view of another embodiment of a frame supporting a sensor cell, pump, and on-off clamps;

FIG. 21 is a partial, perspective view of a sample cell/connector;

FIG. 22 is a cross-sectional view of the sample cell connector of FIG. 21 taken at line 22-22;

FIG. 23 is a schematic view of a sample cell and adapter prior to insertion of the sample cell into the adapter;

FIG. 24 is a schematic view of a sample cell and adapter after insertion;

FIG. 25 is a schematic view of a sample cell, adapter, and extension set;

FIG. 26 is a schematic view of a sample cell and a syringe;

FIG. 27 is a plan view of an extension set system having a layer of material applied thereto that does not transmit red or obscures red;

FIG. 28 is a partial, cross-sectional view of the extension set system of FIG. 27 taken at line 28-28;

FIG. 29 is a plan view of a sample cell with integrated stirrer;

FIG. 30 is a cross-sectional view of the sample cell of FIG. 29 taken at line 30-30;

FIG. 31 is a plan view of a sample cell with a microarray for pathogen identification;

FIG. 32 is a cross-sectional view of the sample cell of FIG. 31 taken at line 32-32;

FIG. 33 is a perspective view of a wired peripheral with sample cell holder;

FIG. 34 is a cross-sectional view of the peripheral of FIG. 33 taken at line 34-34;

FIG. 35 is a plan view of the peripheral of FIG. 33 with an extension set system, the extension set system being separated from the peripheral;

FIG. 36 is a plan view of the peripheral of FIG. 33 with a sample cell associated with the extension set system received within the holder;

FIG. 37 is a perspective view of a wireless peripheral/analyzer with sample cell holder;

FIG. 38 is a cross-sectional view of the peripheral of FIG. 37 taken at line 38-38;

FIG. 39 is a plan view of the peripheral of FIG. 37 with an extension set system, the extension set system being separated from the peripheral; and

FIG. 40 is a plan view of the peripheral of FIG. 37 with a sample cell associated with the extension set system received within the holder.

### DETAILED DESCRIPTION

Although the following text sets forth a detailed description of different embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology or technology developed after the filing date of this patent, which would still fall within the scope of the claims defining the invention.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph.

The present disclosure includes a number of different systems intended to take advantage of the access to the vascular system of a patient that is required to provide intravenous infusion therapy, for example. Using this access, sensing, diagnosis, theranosis, prognosis, and analysis may be readily accomplished with minimum discomfort to the patient. In particular, all of the equipment for performing the infusion therapy and the sensing system may be operated in association with a single insertion/access site. This eliminates the need to create one or more insertion sites, in addition to the infusion therapy insertion site, for the insertion of sensing equipment and/or the aspiration of body fluids, such as blood.

### Internal Sensing System

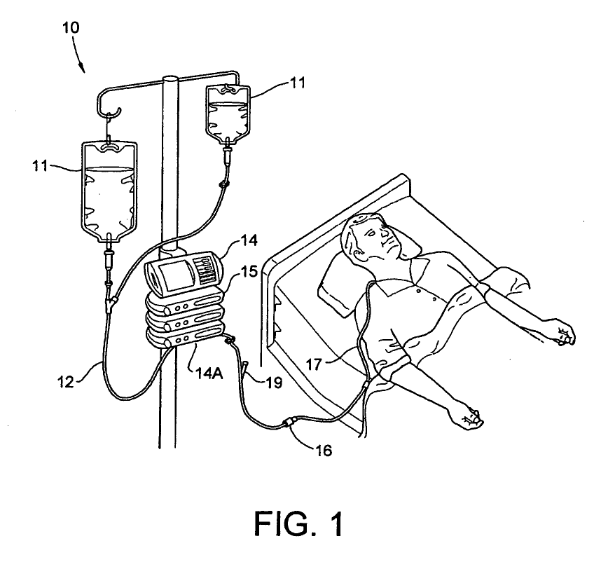

FIGS. 1-9 illustrate a number of examples of a multi-modal catheter sensing system, which is intended to take advantage of the access to the vascular system of a patient that is needed to provide infusion therapy. The sensing may encompass any of a number of analytical techniques that have been developed for testing and identification of the condition of a patient, such as those involving optical and electrical mechanisms. For example, optical sensors may utilize the transmission and the sensing of light to measure glucose, drug levels and/or erythrocytes. Alternatively, electrical sensors may be used to measure cardiac output, viscosity and flow rate. In any event, the access for the system may be made by one of a central venous, mid-line, PICC, peripheral or arterial catheter, for example.

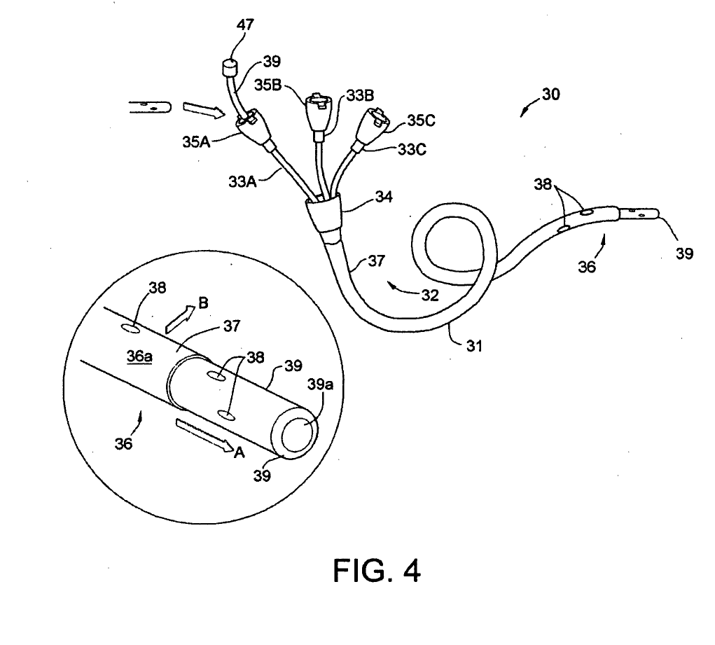

A first example of a system is which may include a multimodal sensing catheter is depicted in FIGS. 1-3. Integrated infusion pump and catheter system 10 includes at least one infusion pump 15 and a controller 14. Tubing 12 connects primary and secondary containers 11 for intravenous administration to the patient. The containers may include saline solution and a medication, or may also include nutrients for administration to the patient. The controller and the infusion pump carefully control and monitor the rate of flow of IV fluid to the patient. Tubing 16 connects the output of the infusion pump(s) to the patient through a multi-lumen central venous catheter 30 (FIG. 4). A luer access device 19 may also be connected in series as shown for immediate delivery of a medication of other substance to the patient.

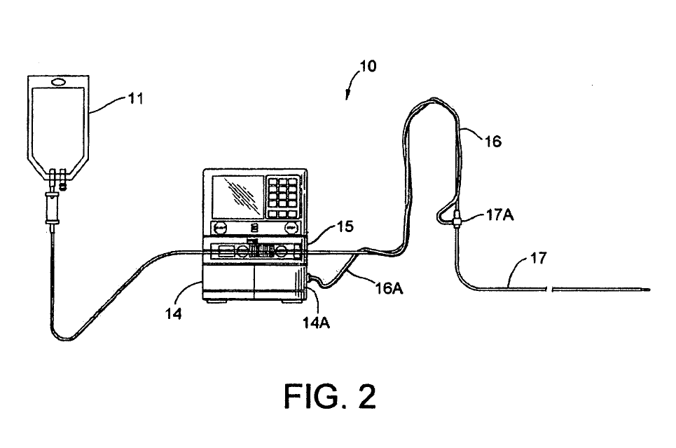

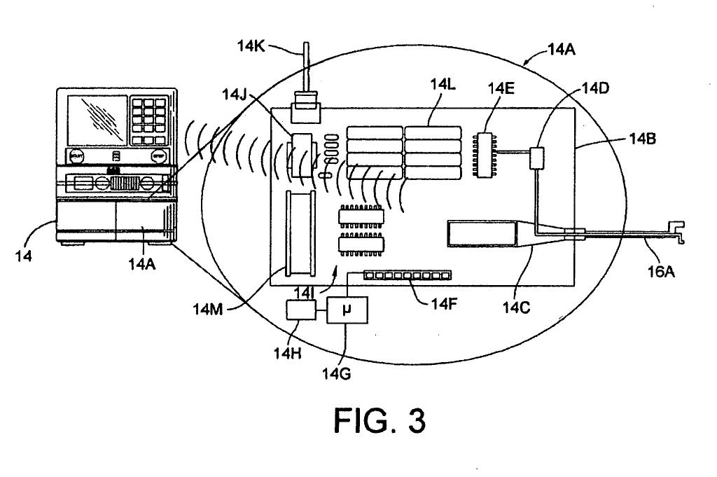

As shown more clearly in FIGS. 2-3, the infusion pump 15 may be associated with a controller 14, a processing module 14A, or both, the collector 14 and the processing module 14A collective referred to herein as a control system. As illustrated, the infusion pump 15, the controller 14 and the processing module 14A are integrated into a single unit. It will be recognized, however, that if the controller 14 and the processing module 14A are not located with or integrated with the infusion pump 15, they may be located elsewhere, such as on the infusion line or a catheter hub, with communication being carried out using wired or wireless protocols, for example. It will also be recognized that the controller 14 and the processing module 14A may be disposed in separate locations.

The processing module 14A may be adapted to interface with optical sensors, electrical sensors, chemical/biochemical sensors, or any combination thereof, in accordance with the type of sensor located in the catheter or infusion line. The processing module 14A may receive the sensing information in the form of an optical or electrical signal, such a light wavelength, a current, or a voltage. The processing module 14A may process the received signal using built-in algorithms, for example, and generate an output that may be transmitted in a format that is clinically useful to the medical practitioner. According to one embodiment, the medical practitioner may make a clinical decision based on the transmitted output, and may operate the controller 14 to change the amount of fluid infused according to the clinical decision made, to provide a “closed loop” delivery system. In another embodiment, the output from the processing module 14A may be sent directly to the controller 14 in a format that will be recognized by controller 14 so as to control/change the amount of fluid that is being infused by the pump 15. These forms of closed loop communication and control may be accomplished using wired or wireless communication protocols.

As illustrated in FIG. 3, the processing module 14A includes the hardware and software to interface with optical sensors, and therefore may be referred to as an optical processing module. The optical processing module 14A includes a plurality of light sources, such as light diodes or light emitting diodes (LED), and also includes circuitry needed for processing an optical detection signal. A fiber optic bundle 16A provides the conduit for transmitting and receiving light signals between the source and the sensor. This fiber optic bundle 16A includes a plurality of optical fibers. Referring briefly also to FIG. 4, the fiber optic bundle 16A interfaces with a connector 47 such as the connector placed on the proximal end of a sensing catheter 39. In another embodiment, the connector 47 may be integrated with the processing module (such as the optical processing module 14A illustrated in FIGS. 2 and 3) which has wireless capability to transmit sensed data (either in raw or processed format) to the controller 14 or to a medical practitioner.

The optical processing module 14A includes a housing 14B, at least one light source 14C (consisting of a laser diode or LED, filter and collimating lenses). The laser light sources are connected to a fiber optic bundle or cable 16A for transmission to a sensor via an optical fiber embedded in the catheter, which will be discussed later. Light is returned using another optical fiber that is also in the optical bundle, and is received by a light collection system 14D (consisting of optical lenses and photomultiplier tube), where the optical signal is converted to an electrical signal, such as voltage. The converted signal is sent to a signal analyzer 14E (consisting of filters and A/D converter), and may then be sent to a data bus 14F for external routing, and may also be processed by an internal CPU 14G. CPU 14G has access to at least one memory 14H. The CPU may perform real time analysis on the data using an algorithm, such as a Fourier transform, to characterize the collected signal.

The result of the analysis may be sent to a medical information system or computing device, and may be displayed thereby to a medical practitioner (such as a nurse). The results may be communicated via a wireless device 14J, such as a radio frequency (RF) output according to a recognized standard, such as Bluetooth® or ZigBee® communications protocols. Alternatively, a wired deice, such as an Ethernet box, may be used to communicate between the processing module 14A and the medical information system or computing device. The processing of the signals from the sensors is virtually instantaneous, so that the medical professional has real-time access to the results of the tests. The processing module may also have other features, such as signal control circuitry 141, for instance, for on/off and timing of the optical signals sent to diagnose the patient. The module may also include batteries 14L, external power supply 14 K, and a cooling fan 14M. Other configurations of an optical module may also be used.

The optical processing module 14A thus includes optical components, backup power (batteries) 14L, a cooling system 14M, and a CPU 14G. The optical processing module 14A also includes software, in the memory 14H or otherwise accessible to the CPU 14G, for manipulating the data, and determining an output, such as a presence or a concentration of an analyte in blood. The optical processing module 14A will preferably share a wireless capability, power source, and a visual display, such as a touch screen, with the pump controller 14. Alternatively, the optical processing module may have separate capabilities, such as a separate visual display that is connected to the module 14A by wires or wirelessly. The fiber optic bundle or cable 16A links the optical processing module 14A with the optical sensors in the sensor catheter 39.

As noted, a sensor catheter allows the healthcare provider to take advantage of the fact that access to a patient's vascular system has already been established for infusion therapy. Since access already exists, no penetration or invasion is necessary and the existing IV catheter may be used.

An embodiment of a multi-lumen catheter, suitable for an infusion therapy is depicted in FIG. 4. Catheter 30 includes a body 31, a proximal portion 32 and a distal portion 36. The illustrated multi-lumen catheter is generally referred to as a triple lumen catheter and defines three lumens that extend to the distal tip 36a. Insertion ports 33A, 33B, 33C, for each of the lumens extend from a proximal end 34 of the catheter which is configured to allow fluid connection to the lumen or for insertion of a catheter, such as sensing catheter 39. As shown in FIG. 4, the tip of the sensing catheter 39 may be inserted through the inlet port 35A and threaded (or using a guidewire) through the catheter until it extends from the tip 36a of the multilumen catheter 30. The multi-lumen catheter 30 and sensing catheter 39 may be coated with an antimicrobial or anticoagulant material. One or more of the other two remaining lumens 33B and 33C may then be used for infusion therapy (i.e., be in fluid communication with the infusion pump 15 and the patient).

Sensing catheters 39 used for the embodiments herein may be made of silicone or polyurethane, or other medically acceptable polymer or blend of polymers. The catheters should be as thin as possible, preferably not more than ⅛″ in diameter, about 3-9 Fr. Optical fibers are very thin, typically about 100-500 micrometers, usually with optical cladding, so it is possible to make the catheter embodiments with very narrow diameters. The catheters may be made by extrusion or any other suitable manufacturing technique, for later insertion of the sensors and other parts of the integrated sensor and infusion pump or other device. If the catheter is constructed with optical sensors, the optical fibers may be integrated with the catheter in certain embodiments. As a consequence, the optical fibers/cables, wires/leads, electrodes, etc. may be within the catheter either by being disposed in a lumen of the catheter or by being embedded in a wall of the catheter, for example.

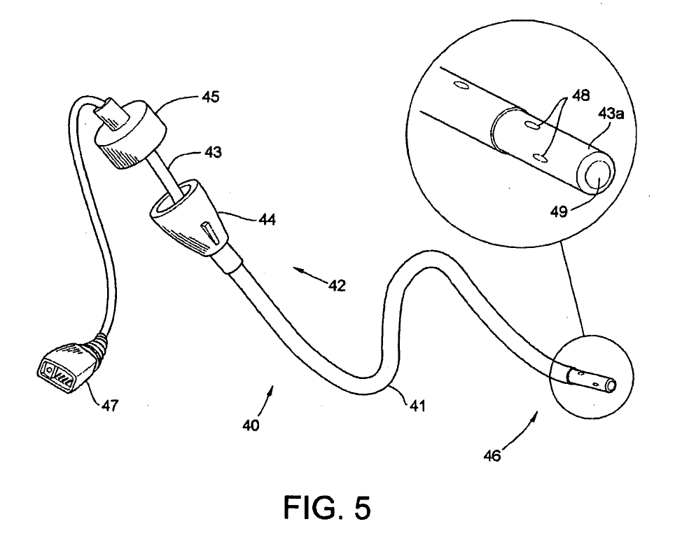

As will be seen below in FIG. 5, a sensor bundle 43 may include an electrical, chemical, or biochemical sensor at its distal end. However, modifications to sensor catheters are required to account for these other modes of sensing. For example, an electrical mode of sensing may involve replacing fiber optic cable with an electrical cable or lead, emitter and collector fiber optic tips with anode, cathode and reference electrodes. On the other hand, chemical or biochemical sensors may include a basic electrical or optical sensor where the fiber optic tips or electrodes are surface-modified with suitable molecules for the purpose of sensing (e.g., a chemical or biochemical reaction may occur that generates an electrical or optical signal via a electrochemical or photochemical reaction); this sensor may have an array format similar to that illustrated in FIGS. 31 and 32, for instance. For purposes of this paper, sensors in which signals passed are primarily electrical are called an electrical sensor, to distinguish them from optical sensors.

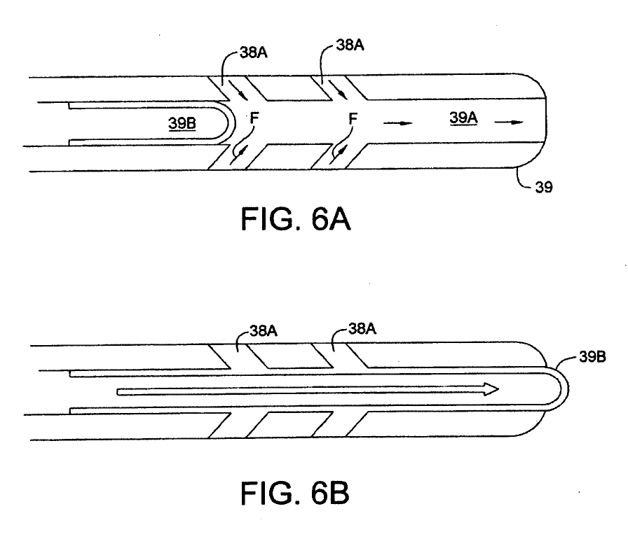

FIGS. 6A-6B depict a cross-sectional view of the sensing catheter 39. The catheter 39 includes inlet side ports 38A which extend from the exterior of the catheter at an angle into the central lumen 39A. The catheter 39 is placed with the tip pointing downstream of the blood flow such that the blood flows into the side holes ports 38A. From the side ports 38A the blood flows into the lumen 39A as shown by arrows F. Moreover, the blood flows through the apertures with a laminar flow which reduces noise and thereby facilitates measurement of the desired parameter. In addition, one may change the optical length by varying the diameter of the central lumen 39A.

The sensing catheter 39 includes a diaphragm 39B, such as an inflatable balloon or occluder, which acts to prevent blood from flowing further proximally into the lumen. Alternatively, a hydrophobic valve may be used to prevent blood or other fluid from flowing from the access point.

If blood is found to clot in the lumen 39A or at side ports 38A, or when the diagnostic procedure has been completed, and the medical professional is ready to finish, the diaphragm 39B may be inflated or advanced distally within the central lumen in connection with a source of air attached thereto, for example. As shown in FIG. 6B, the diaphragm 39B will clear the central lumen 39A and ports 38B of any clots. Furthermore, this procedure will cause as little blood as possible to remain within the catheter 39, thus limiting the likelihood of occlusion.

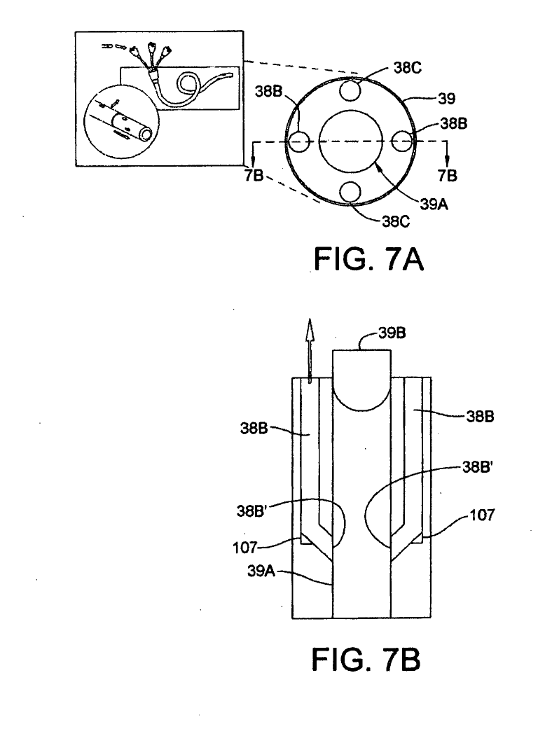

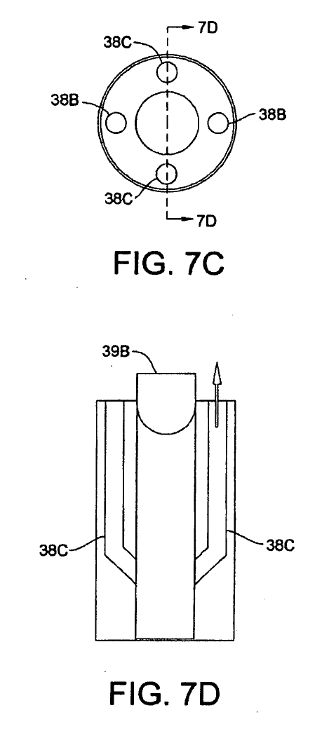

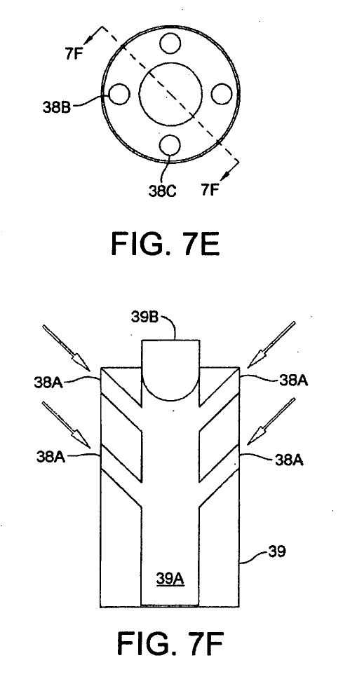

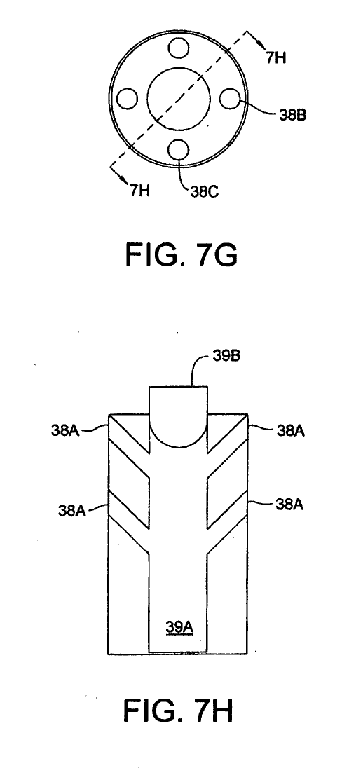

FIGS. 7A-7H depict a plurality of cross-sections of the optical sensing catheter 39, that is, a catheter adapted specially for use with optical sensors. This particular catheter also has additional lumens to accommodate electrical sensors. In this series of figures, FIGS. 7A, 7C, 7E and 7G represent axial cross-sections, with FIGS. 7B, 7D, 7F and 7H representing the corresponding views transverse to the axial cross sections.

In FIGS. 7A and 7B, catheter 39 includes the central lumen 39A and an occluder, such as balloon 39B, as explained above. There are also two optical sensing fibers 38B. The sensing ends 38B′ are separated by one hundred and eight degrees for accommodating portions of an optical system, e.g., one end 38B′ for the emission of an optical signal or light and one end 38B′ for collecting of the resulting light. Referring to FIGS. 7C and 7D Catheter 39 also has additional electrical sensors 38C. Generally such an electrical sensing system requires three electrodes, an anode, a cathode and a reference electrode for the electrical measurements. Generally the anode and cathode will be separated from the optical sensing ends 38B′ (FIG. 7B) by ninety degrees. In FIGS. 7E and 7F, and also in FIGS. 7G and 7H, side ports 38A are seen to intersect with the main lumen 39A, allowing blood to flow through the side ports, near the sensors, and allowing analysis to take place.

The optical systems described in FIGS. 1-7 may be termed as single-mode optical systems, in that is utilized if one mode of detection is desired. For instance, light from one of the laser diodes may be used to fluoresce analyte species, while the collecting sensor collects the light that is emitted as a result of the fluorescing. The optical processing module then routes the collected light to a photomultiplier tube for amplification and classification according to wavelength of light emitted. The result is then processed electronically to determine the quanta emitted and ultimately, to determine a presence and a concentration of a particular analyte or class of analytes. In other embodiments, light of a particular wavelength may be used in a simple incidence/absorption analysis. These are examples of single-mode optical analysis.

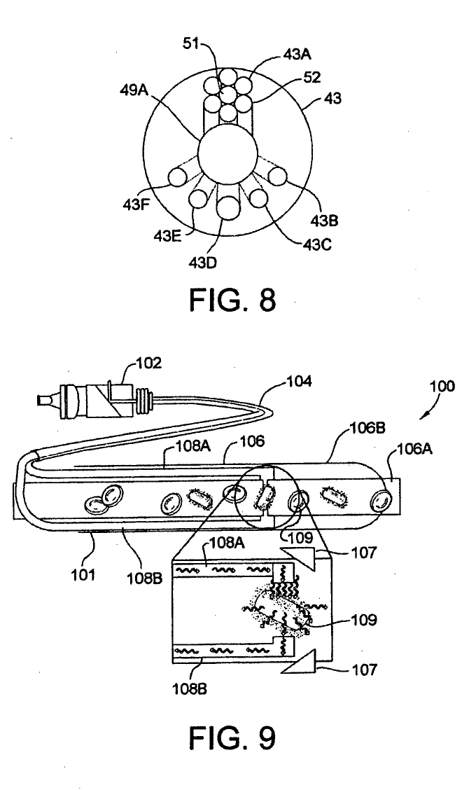

With the integrated system described herein, more sophisticated optical analysis may also be performed. For example, multi-mode optical sensors may be used in the embodiment of FIG. 8. Catheter 43 includes a central lumen 49A. A 6+1 cluster 43A of optical fibers extend along one side of the central lumen 49A. The cluster 43A includes a central fiber 51 that serves to emit light into the blood in the lumen. The surrounding fibers 52 serve as collection fibers. Opposite the cluster 43A on the other side of the lumen 49A and spaced generally equally are a plurality of additional fibers 43B, 43C, 43D, 43E and 43F. These fibers serve to collect light that has been emitted from the emitting fiber 51 and has interacted in the blood. Referring also to FIG. 7F, catheter 43 is similar to catheter 39 and includes side ports 38A to provide for blood flow along the central lumen 49A and along the sensor ends 43A-43F. In all the above embodiments, suitable mirrors (such as identified as 107 in FIG. 7B) may be integrated with the fiber optic cables so that the light can be focused normally from the sides of the catheter main lumen 39A. 49A.

In this embodiment, the sensor ends of the collecting fibers 52, 43B, 43C, 43E, and 43F can collect light that has been scattered by the blood and transmit the collected light to the module for processing and analysis. The sensor ends of the collecting fibers 52 can collect light that has been reflected by the blood and transmit the collected light to the module for analysis. The sensor end of fiber 43D can collect light which has been transmitted through the blood, and the fiber can transmit this collected light to the module for transmission/absorption analysis. The sensor ends of all the fibers except the emitting fiber 51 can collect light that fluoresces from the blood or analytes in the blood and transmit the collected light to the module 14A for analysis. The light collected by the sensing ends of the fibers 43B-43F may also be analyzed to determine the scattering of the light by the blood sample. Thus this embodiment provides for the ability to collect emitted light after it has been scattered, reflected, transmitted and absorbed by the blood sample. In addition this embodiment allows for the collection and analysis of light that has been fluoresced by the blood sample.

The plurality of receptors allows the detection of patterns, shapes, and multiple other characteristics of the blood and the species in the blood due to the interaction of light. One additional example of how a particular mode is used is depicted in FIG. 9. The catheter 100 includes a catheter body 101, a proximal connector 102, a proximal portion 104 and a distal portion 106. The catheter has a main lumen 106A, and a light source optical fiber 108A and light collector optical fiber 108B. The light may be reflected and subsequently guided from source fiber 108 by mirror 107, so that the light is transmitted normally through the blood and into analyte 109, causing analyte 109 to fluoresce. The fluorescence, of a different and higher wavelength, is detected by detector optical fiber 108B. The detected fluorescence is then transmitted by the optical fiber 108B through connector 102 and to an optical processing module.

The above detection scheme may be accomplished with the embodiments as described and with a single or multiple wavelengths of light. The light may be continuous or pulsed.

Referring to FIG. 5, a second embodiment of a system for infusion therapy along with use of a sensing catheter 43 is illustrated. The sensing catheter 43 is inserted through a single lumen catheter 40 until a tip 43a extends from the distal end of the catheter. In this embodiment the optical sensing and electrical sensing elements may take any of the forms described above. However, the catheter 43 is configured so that infusion therapy is conducted through intermittent use of a lumen 49 formed within the catheter. Such a system is not preferred, as it requires stoppage of the infusion therapy during measurement of the blood parameters through use of the sensing catheter 43 as described above.

Referring back to FIG. 4, although the embodiment described therein was described as the sensing catheter 39 being separate from the multi-lumen catheter 30, it is also envisioned that the sensing catheter may be formed integrally with the catheter 30. While examples of the systems illustrated in FIGS. 1-9 all included a sensor that is, in whole or in part, disposed in a patient's vein, similar embodiments may be envisioned for use in an arterial access site as well.

### Infusion Line-Based Sensing Systems

While the examples of the systems illustrated in FIGS. 1-9 all involved a sensor that is, in whole or in part, disposed in a patient's vein or artery, the disclosure of the present application is not so limited. Rather, other systems may be designed that also are intended to take advantage of the insertion site and/or administration set used during infusion therapy to connect the patient to a fluid source (IV bag, cassette, etc.). According to such systems, the bodily fluids are drawn from the patient into a sample cell, for example, and then the bodily fluids in the cell are analyzed.

For example, the sample cell could be designed as part of an IV administration set used for infusion therapy, or to be connected in-line with such an administration set. In either event, the cell is disposed so that it is outside the patient's body, but so that it is in fluid communication with the patient's vein or artery via a catheter. Blood may be drawn into the cell by reversing the action of an infusion pump associated with the administration set, or by actuating or operating a separate device that draws blood into the sample cell. The blood drawn into the sample cell from the vein may be flushed out of the sample cell afterward by passing fluids through the administration set into the patient.

As another example, the sample cell could be designed to receive blood drawn from the patient, either directly or indirectly, from the insertion site used with the administration set. That is, the administration set may be detached from the catheter hub, and the sample cell could be connected to the hub instead, via an adapter according to certain exemplary embodiments. In this embodiment, the analysis may be performed while the sample cell is still attached to the catheter hub. Alternatively, the blood may be drawn into a syringe or vacutainer, for example, and then transferred into the sample cell.

The sensors associated with the sample cells according to any of these embodiments may be formed integrally with (i.e., as one unit) or attached to the sample cells, similar to the embodiments discussed in FIGS. 1-9, above. However, as illustrated in FIGS. 11-14, for example, the sensor may be assembled as part of a device that is associated with the sample cell, but not formed with or attached to sample cell; as illustrated, the sample cell is received in a receptacle or recess in the device. This associated device may include the necessary interface and processing capabilities to analyze the blood in the sample cell and signal the user, for example, to the presence of a pathogen in the material (e.g., blood) in the sample cell. The user may receive visual indication, an aural indication, or a combination of the two, for example.

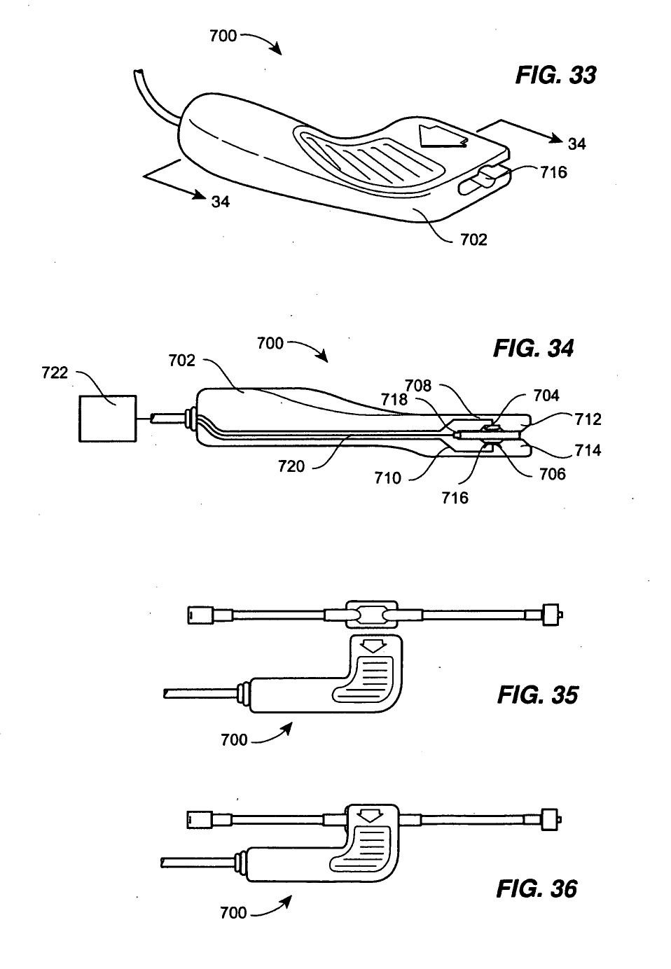

According to other embodiments, the associated device may include only interface capabilities, and be coupled to or be capable of being coupled to a further device that includes the remaining interface, processing and signaling capabilities. As seen in FIGS. 33-40, the device may be a peripheral having a receptacle or recess formed to accept at least the sample cell and certain interface capabilities, but lack the processing and signaling capabilities. The peripheral device may be associated with a device that includes these processing and signaling capabilities, which association may be in the form of a hard-wired connection or a wireless connection, for example. The peripheral device may be incorporated into a handheld platform that may interface directly with the sample cell or be incorporated to an associated pump (as a processing module) that may be connected by a harness or bus, or wirelessly.

It will be recognized, that the various examples of the sample cell discussed below may be combined with the various examples of the interface/processing/signaling device or system to define a variety of different sensor or sensing systems. The illustrated embodiments below are thus exemplary of such combinations, but not exhaustive of all of the possible combinations. One skilled in the art will appreciate that other combinations may be formed by associating the various sample cells and interface/processing/signaling devices to define a sensing system. Moreover, where variants are described in regard to these embodiments, it will be recognized that the variants are not limited merely because they are discussed with respect to one particular example of a broader group or class of related devices.

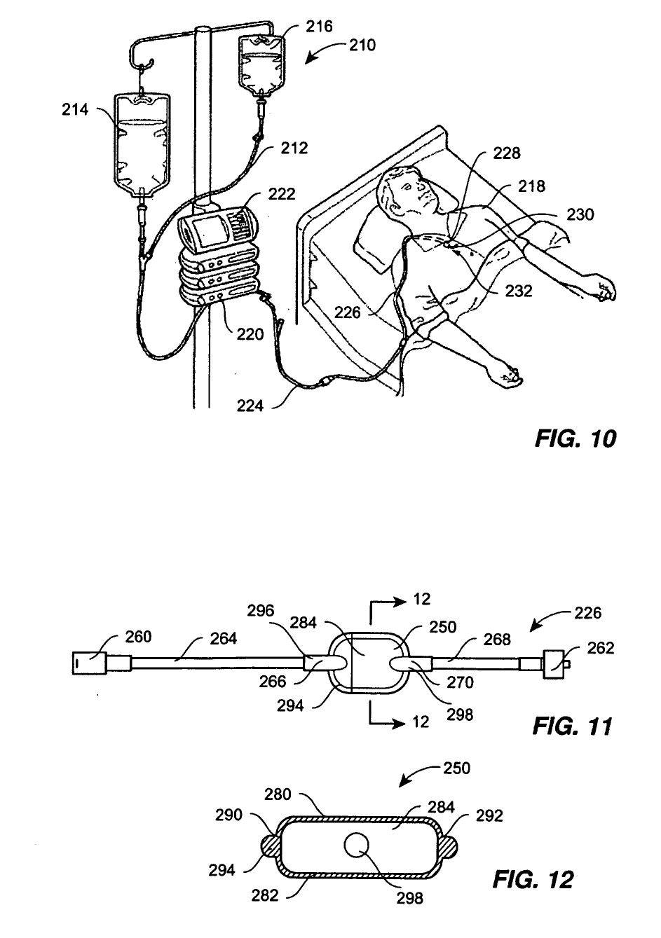

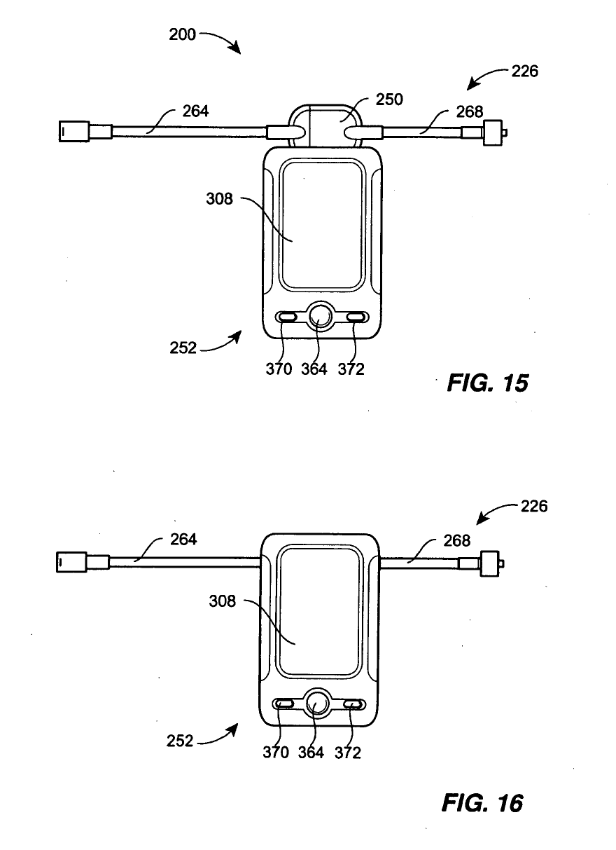

FIGS. 10-16 illustrate a first embodiment of a sensor system 200 (more particularly seen in FIG. 15) wherein the bodily fluids (e.g., blood) are drawn from the patient to a site remote from the vein. As illustrated in FIG. 10, the sensor system may be used with an infusion system 210 that includes an administration set 212. The administration set 212 is connected to primary and secondary containers 214, 216 for intravenous administration to a patient 218. The containers 214, 216 may include saline solution and a medication, or may also include nutrients for administration to the patient 218. The infusion system 210 may also include at least one infusion pump 220 and an associated pump controller 222 through which tubing 224 of the administration set 212 passes. The infusion pump 220 and the controller 222 carefully control and/or monitor the rate of flow of fluid from the containers 214, 216 to the patient 218. The administration set 212 is connected to an extension set 226, which is in turn connected to a connector hub 228 of a intravenous catheter 230 that has been run into the patient 218 via an insertion site 232

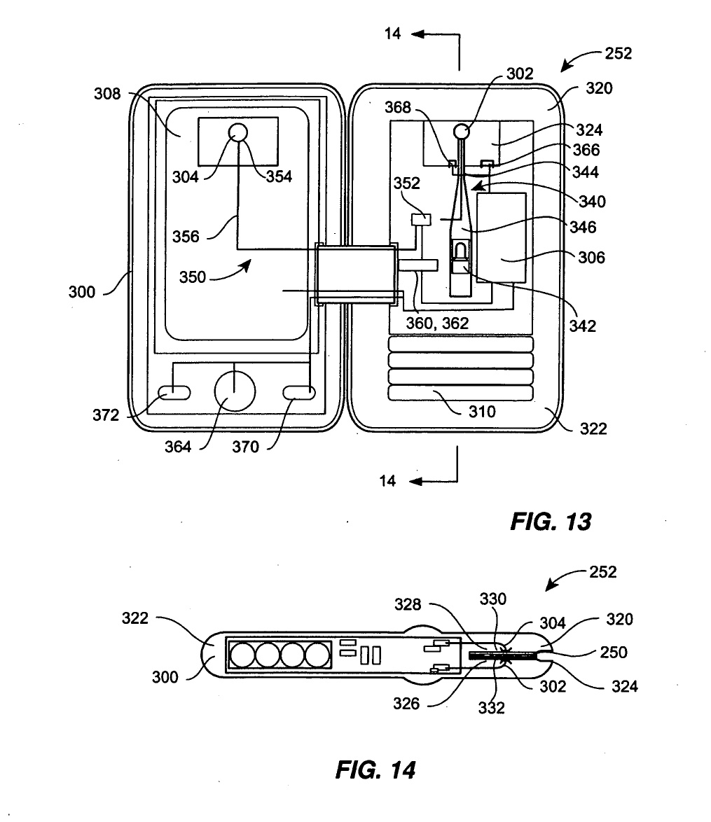

According to this embodiment, the sensor system 200 includes a sample cell 250 (see FIGS. 11 and 12), an analyzer 252 (see FIGS. 13 and 14), and the infusion pump 220 and administration set 212 that are also part of the infusion system 210. In particular, the sample cell 250 is connected to the extension set 226, and is preferably integral (i.e., permanently attached or integrated with) the extension set 226. The sample cell 250 is received into the analyzer 252 (see FIGS. 15 and 16) so that the bodily fluids in the sample cell 250 may be subjected to one or more sensing modes, and so that the analyzer may determine a physical condition of the patient thereby. The infusion pump 220 (and associated administration set 212) is used to draw bodily fluids (e.g., blood) into the sample cell 250 at least during the time the sample cell 250 is received into the analyzer 252, and to flush the sample cell 250 with infusion fluid afterward.

Starting then with FIGS. 11 and 12, the sample cell 250 is formed integrally with the extension set 226 as illustrated. In particular, the extension set 226 includes an administration set connector 260 and a catheter hub connector 262. As illustrated, the administration set connector 260 is a female luer, while the catheter hub connector 262 is a male luer. A first length of tubing 264 connects the administration set connector 260 to a first end 266 of the sample cell 250, while a second length of tubing 268 connects the catheter hub connector 262 to a second end 270 of the sample cell. Consequently, the sample cell 250 is connected between the administration set connector 260 and the catheter hub connector 262. The tubing 264, 268 may be connected to the sample cell 250 as well as the connectors 260, 262 using solvent bonding methods for example.

In particular, the sample cell 250 includes a pair of opposing walls 280, 282 spaced from each other to define a test region 284 therebetween (see FIG. 12). While the material used to form the walls 280, 282 may come from a variety of sources, at least one of the opposing walls 280, 282 may be defined by quartz or ultraviolet-grade fused silica, either in whole or in part. Quartz or fused silica may reduce the background signal, as well as limiting or eliminating auto-fluorescence that occurs when other materials are used for the walls 280, 282. The reduction or elimination of such interference may be combined with an increased transmission of the light of the appropriate wavelength, leading to a better signal-to-noise ratio for the sensor system when quartz and/or fused silica is used.

The cell 250 has an inlet defined by the second end 270, which inlet is in fluid communication with the test region 284. According to the illustrated embodiment, the cell also has an outlet defined by the first end 266. In this regard, the convention of “inlet” and “outlet” is used wherein the flow is defined according to the operation of the sample cell 250 when sensing and analysis is being performed. It will be recognized, that fluid can and does actually flow from “outlet” to “inlet” during other modes of operation of the sensor system 200 and the infusion system 210.

It will be recognized with reference to FIGS. 11 and 12 that while the first end 266 and the second end 270 of the sample cell 250 define passages that are primarily circular or elliptical in cross-section (as seen in FIG. 12), the shape of the passage in the vicinity of the test region 284 is rectangular in cross section (as also seen in FIG. 12). In particular, the opposing walls 280, 282 are substantially planar in shape, at least in the vicinity of the test region 284, the opposing walls 280, 282 being closed at opposing edges 290, 292 by a boundary wall 294 that extends about the periphery of the planar walls 280, 282. The boundary wall 294 has circular or elliptical bores 296, 298 (which may be referred to as tubing bond pockets, when used in that fashion) that define the passages in the first and second ends 266, 270 of the sample cell 250 that receive the tubing 264, 268. The boundary wall 294 therefore also defines the transition regions between the passages of circular or elliptical cross-section and the test region 284 of rectangular cross-section.

It will be further recognized that the distance between the opposing walls 280, 282 in the test region 284 may be smaller than the diameter of the passages in the first or second ends 266, 270 of the sample cell. In fact, as illustrated, the walls 280, 282 each have a length and a width. The opposing planar walls 280, 282 are spaced apart by a distance that is at least an order of magnitude smaller than the length and the width of the walls 280, 282.

Referring next to FIGS. 13 and 14, as well as FIGS. 15 and 16, the analyzer 252 is illustrated therein. According to this example of the sensor system 200, the analyzer 252 includes the necessary hardware and software (or firmware, for that matter) required to perform the sensing and analysis required by the system 200. According to the illustrated embodiment, the analyzer 252 includes the necessary hardware and software to perform an optical sensing of blood in the sample cell 250 and an analysis of the blood to determine the presence (or absence) of a pathogen in the blood, and thus a physical condition of the patient (e.g., presence or absence of a blood stream infection). This sensing may involve detection of light in one or more of the following modes: fluorescence, absorption, transmission, reflection, scattering, and polarization. The sensing could instead involve other properties of light, and could even be non-optical (e.g., electrical).

To the extent that the sensing may involve electrical sensors such as are described in greater detail above, instead or in addition to optical sensor, the sample cell may have one or more contacts and/or one or more electrodes formed in the walls of the cell, which contacts may be coupled to contacts mounted to or on the analyzer. In addition, the analyzer would include hardware and software to interface with the electrical sensor or sensors used. For example, the analyzer may include contacts in the sample cell holder to connect electrical input and output circuitry to the sample cell. An electrical power controller and function generator may be included in the analyzer, with electrical wiring or tracing replacing the optical fibers in the illustrated embodiment. A potentiometer, amperometer, electrical conductometer, or other electrical equipment may be used to process the electrical signal.

Returning to the illustrated embodiment, the analyzer 252 includes a housing 300, a light emitter 302, a light receptor 304, a processor 306, a signaling device 308, and an on-board power supply 310. As illustrated in FIGS. 12 and 13, the light emitter 302, the light receptor 304, the processor 306, and the signalizing device 308 are all mounted on or in the housing 300, which may be hand-held. It will be recognized that one or more of these structures (302, 304, 306, 308) may be disposed in housings other than the housing 300; further discussion of such examples is deferred to FIGS. 33-40, below. The housing 300 has a first end 320 and a second end 322.

The first end 320 of the housing 300 includes a holder 324, in which at least the test region 284 of the sample cell 250 is received at least during analysis of the blood in the sample cell 250. The holder 324 may include opposing walls 326, 328 that are spaced from each other to define a space therebetween in which the sample cell 250 is received (see FIGS. 15 and 16). The distance between the walls 326, 328 is selected so that it is slightly larger than the thickness of the sample cell 250. The walls 326, 328 may extend beyond the periphery of the boundary wall 294 that is disposed about the opposing walls 280, 282 of the sample cell 250 (see FIG. 16). After this fashion, the holder 324 limits the possibility of light external to the analyzer 252 interfering with the operation of the light receptor 304. As shown in FIG. 16, the walls 326, 328 may extend substantially further than the periphery of the boundary wall 294, such that portions of the tubing 264, 268 may also be covered by the walls 326, 328 as well. The walls 326, 328 may have opposing surfaces 330, 332 (see FIG. 14) that correspond to the external surfaces of the sample cell 250 and tubing 264, 268 so as to ensure that light from the surroundings does not interfere with operation of the light receptor.

The light emitter 302 and the light receptor 304 are disposed about the holder 324 adjacent to the test region 284 when the sample cell 250 is disposed in the holder 324. In particular, as illustrated in FIG. 14, the light emitter 302 and the light receptor 304 are spaced from each other, with the light emitter 302 mounted on one of the walls 326 and the light receptor 304 mounted on the other of the walls 328. As a consequence, with the sample cell 250 received within the holder 324, the opposing walls 280, 282 of the sample cell 250 are disposed between the light emitter 302 and the light receptor 304 such that the light emitter 302 and the light receptor 304 are on opposing sides of the test region 284.

It will be recognized that the light emitter 302 and the light receptor 304 are each assemblies that generate light of one or more wavelengths, and that receive light. As illustrated, the light emitter 302 and the light receptor 304 are on opposing sides of the test region 284. However, according to other embodiments, the emitter 302 and receptor 304 may be on the same side of the test region 284. Further, whether the emitter and receptor are referred to as being on opposing sides or the same side, the reference may only be true as to those parts or sections of the assembly in the immediate vicinity of the test region 284.

As to the assemblies that generate and receive light, and thus of which the light emitter 302 and light receptor 304 are a part, these assemblies may be referenced in FIG. 13. In particular, the light generation assembly 340 may include a light source 342, such as a light emitting diode or a laser diode. The light generation assembly 340 may also include optical cables, splitters, lenses, mirrors, filters, grids, etc. that connect the light source 342 to the light emitter 302; the light emitter 302 including a colliminating lens and one such optical cable 344 connected to the light source 342 by intermediate mirrors and lenses 346. For that matter, the light emitter 302 and the light source 342 may be one in the same structure according to certain embodiments. Likewise, the light reception assembly 350 may include a photomultiplier tube (PMT) 352, which generates an electrical signal (e.g., a voltage signal) in response to the light energy received thereby. Here as well, the PMT 352 may be coupled operatively to the light receptor 304 by optical cables, lenses, mirrors, filters, etc.; for example, the light receptor 304 may be defined by a lens 354 that focuses the light received into an optical cable 356 coupled to the PMT 352.

It will be further recognized that the light emitter 302 and light receptor 304 are not limited to a particular mode of operation. For example, the light emitter 302 may emit light of a particular wavelength that makes certain organisms or analytes in the blood fluoresce; for example, certain pathogens fluoresce when excited with light from the uv-vis portion of the spectrum. However, the light emitter 302 and the light receptor 304 may be used instead to measure absorption, reflection, polarization, or transmission. As such, according to other embodiments, the light emitter 302 and light receptor 304 may in fact be disposed on the same side of the sample cell 250, instead of with the sample cell 250 disposed between the light emitter 302 and the light receptor 304.

The signal from the light receptor 304, or more particularly the PMT 352, is received by the processor 306. Again, it will be recognized that one or more interface circuits may be disposed between the processor 306 and the light receptor 304, while the processor 306 may still be considered operatively coupled to the light receptor 304 to receive a sensor signal therefrom. As illustrated, the PMT 352 may be operatively coupled via a filter 360 (which may be a band-pass filter) and an analog-to-digital converter 362 to the processor 306.

The processor 306 may be programmed to carry out an algorithm or program that determines the presence (or absence) of a pathogen in the blood of the patient in accordance with the sensor signal received from the light receptor 304 (via PMT 352). From this determination, the processor 306 may be further programmed to determine a physical condition of the patient, such as the presence or absence of a blood infection. The processor 306 may be further programmed to operate the signaling device 308 in accordance with the determination made either as to the pathogen or the physical condition.

The signaling device 308 may be operatively coupled to the processor 306 to receive a processor signal therefrom. The signaling device 308 may then provide an indication associated with the physical condition of the patient according to the processor signal. The signaling device 308 may do so visibly, by actuating a light emitting diode, for example. As illustrated, the signaling device 308 may include a display, such as a liquid-crystal display (LCD). The signaling device 308 may do so aurally, by actuating a buzzer or other sound generator. The signaling device 308 may do so both visibly and aurally. Alternatively, the signaling device 308 may provide a signal to a remote site, wirelessly for example, to notify a person or a computer located at the remote site as to the determination made by the processor 306.

The system 200 is operated in the following fashion. Initially, the medical practitioner will stop the infusion pump 220, thereby stopping any infusion through the extension set 226 that includes the sample cell 250. The sample cell 250 is then placed within the holder 324 of the analyzer 252. The practitioner may then actuate an input device (such as button 364), which sends a signal to the processor 306 to start the analysis. The processor 306 also checks a sensor 366 (such as a proximity switch) to determine that the sample cell 250 is fully and properly engaged in the holder 324.

At this point, the processor 306 may send a signal, over a hardwired or wireless connection, to the pump 220 or the pump controller 222 to reverse the flow of the fluid through the extension set 226. The processor 306 will continue to control the pump 220 to reverse the flow of fluid through the extension set 226 until blood (or other bodily fluid of interest) fills the sample cell 250. A sensor 368 may be provided that provides a signal in response to detection of whole blood (or bodily fluid) in the sample cell 250. When the signal is received from the sensor 368, the processor 306 signals the pump 220 to cease operation. Alternatively, these steps may be carried out by the practitioner manually by operating the pump controller 222 to achieve the same result. An on-off clamp may be used with the administration set 212 on the side of the pump 220 between the containers 214, 216 and the pump 220.

The processor 306 may then activate the light source 342 automatically (i.e., without further input from the practitioner), or the practitioner may depress an input (the button 364 or one of buttons 370, 372) to signal to the processor 306 to activate the light source 342. A condition is detected by the PMT 352 (which receives a light input via the light receptor 304), which provides a signal to the processor 306. Based on the results, the processor 306 may signal the pump 220 to resume operation, may delay or terminate operation of the pump 220, may store the results of the analysis, and/or may cause a signaling device 308 to actuate to alert a medical practitioner acting as caregiver to the patient. According to the embodiments where the practitioner operates the pump 220 manually, the practitioner would carry out the steps necessary to resume infusion (change the on/off clamp, actuate the pump, etc.) after consulting the signaling device 308.

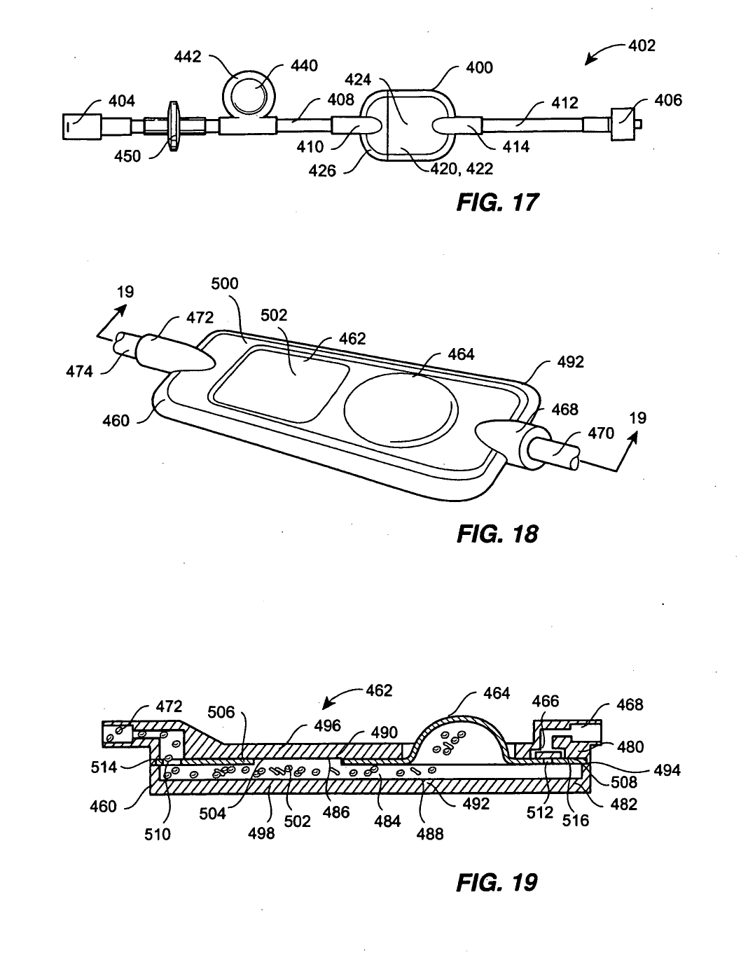

Having thus discussed one non-intravenous sensor system including sample cell and analyzer, other variants of the sample cell will be discussed. In particular, as was the case in the preceding example, the sensor system utilized the infusion pump associated with the infusion system as a mechanism for drawing bodily fluids, such as blood, into the sample cell. Turning next to FIGS. 17-20, several variants of the sample cell 250 are illustrated, which variants do not require the use of an infusion pump for drawing the bodily fluids (e.g., blood) into the sample cell. Such variants may be used with the analyzer 252 discussed above, with the necessary changes being made in regard to the geometry of the holder, for example, to accommodate the differences in placement or shape of the sample cell.

For example, a first variant is illustrated in FIG. 17, which variant includes a sample cell 400 is integral with the extension set 402. As in the example illustrated in FIGS. 11 and 12, the extension set 402 includes an administration set connector 404 and a catheter hub connector 406. As illustrated, the administration set connector 404 is a female luer, while the catheter hub connector 406 is a male luer. A first length of tubing 408 connects the administration set connector 404 to a first end 410 of the sample cell 400, while a second length of tubing 412 connects the catheter hub connector 406 to a second end 414 of the sample cell 400. Consequently, the sample cell 400 is connected between the administration set connector 404 and the catheter hub connector 406.

Similar to the embodiment of FIGS. 11 and 12, the sample cell 400 includes a pair of opposing walls 420, 422 spaced from each other to define a test region 424 therebetween. Likewise, a boundary wall 426 is disposed about the periphery of the walls 420, 422, and defines ports at the ends 410, 414 to accept the tubing 408, 412. However, as mentioned above, the extension set 402 also includes other structures that permit the blood to be drawn into the sample cell 400 without the use of an infusion pump. A pump may still be included for other purposes, however, such as to infuse fluid to patient in a controlled fashion.

In particular, the extension set 402 includes a flexible diaphragm 440. The diaphragm 440 may be attached to a housing 442 that is connected to the tubing 408 that connects the administration set connector 404 to the first end 410 of the cell 400. The diaphragm 440 is thus disposed between the administration set connector 404 and the sample cell 400. The diaphragm 440 and the housing 442 may define a flash bulb, for example.

The diaphragm 440 is moveable between a depressed state and a distended state. In the depressed state, fluid is ejected from the extension set 402. In the distended state, which follows the depressed state, blood is drawn into the extension set 402 via the intravenous catheter into the sample cell 400. The diaphragm 440 may draw sufficient blood into the sample cell 400 during a single cycle (depressed stated/distended state) to fill the sample cell 400 and permit sensing and analysis using an analyzer similar to that illustrated in FIGS. 13 and 14. However, it may be necessary to repeat the cycle several times to drawn blood into the sample cell under other conditions.

It will be further recognized that the extension set 402 may include a one-way valve 450. The one-way valve 450 is disposed between the administration set connector 404 and the diaphragm 440 through its placement in the tubing 408 that connects the administration set connector 404 to the sample cell 400. The one-way valve 450 is open to permit fluid to flow in the direction from the administration set connector 404 to the catheter hub connector 406. By contrast, the one-way valve 450 is closed to limit flow in the direction from the catheter hub connector 406 to the administration set connector 404. Alternatively, an on/off clamp may be used, as discussed above relative to the embodiment of FIGS. 10-16.

A further variant is illustrated in FIGS. 18 and 19. According to the illustrated variant, a frame 460 is provided, a sample cell 462, a diaphragm 464, and a one-way valve 466 being attached to the frame 460. The frame 460 has a first port 468 that may be coupled to an extension set connector via tubing 470, and a second port 472 that may be coupled to a catheter hub connector via tubing 474. However, according to still other variants, the first and second ports 468, 472 may define the extension set connector and the catheter hub connector, respectively; such a modification would permit the variant to be connected between an extension set and the catheter hub, or even between an administration set and the extension set.

As seen in cross-section in FIG. 19, the frame 460 includes a first housing section 480 and a second housing section 482, which housing sections 480, 482 are joined together to define a fluid path 484 between the first port 468 and the second port 472. In particular, the first port 468, 472 are defined in the first, or upper, housing section 480, while the fluid path 484 is defined by opposing surfaces 486, 488 of opposing walls 490, 492 of the first and second housing sections 480, 482, respectively. The first and second housing sections 480, 482 may be joined together about a peripheral edge 494 by ultrasonic welding, for example.

The first housing wall 490 and the second housing wall 492 define the sample cell 462. In particular, a section 496 of the wall 490 and a section 498 of the wall 492 define the sample cell 462. This may be illustrated on an outer surface 500 of the first housing section 480 by etching a border or boundary corresponding to the sample cell 462, and in particular the test region 502 (see FIG. 18). Alternatively, a label may be disposed on the outer surface 500 to define the periphery of the cell 462 and/or the test region 502. Furthermore, the sample cell 462 and/or test region 502 may be circumscribed within the frame 460 by a window 504 defined in a plate 506 disposed between the first and second housing sections 496, 498.

The plate 506 may be disposed between the first and second housing section 496, 498 such that a peripheral edge 508 of the plate 506 is disposed between the first and second housing sections 496, 498. With the first and second housing sections 496, 498 attached together, the plate 506 is held in position. In addition to the window 504 formed in the plate 506 to permit light to pass into the test region 502, the plate may have apertures 510, 512 formed at ends 514, 516 to permit access between the ports 468, 472 and the fluid path 484.

The plate 506, or more particularly a section or region thereof, also defines the diaphragm 464. As illustrated in FIG. 19, the diaphragm 464 is formed integrally (i.e., as one piece) with the remainder of the plate 506. It will also be recognized that the plate 506 could instead have had an opening formed therein, and the diaphragm formed therethrough, through the use of two-shot molding processes for example. The diaphragm 464 operates to draw blood and/or other bodily fluids into the test region of the sample cell 462.

Attached to the plate 506 is the one-way valve 466. In particular, the one-way valve 466 is positioned between the port 472 and the diaphragm 464. The valve 466 operates to control the flow of fluid through the fluid path 484 between the ports 468, 472 in a fashion similar to the example illustrated in FIG. 17.

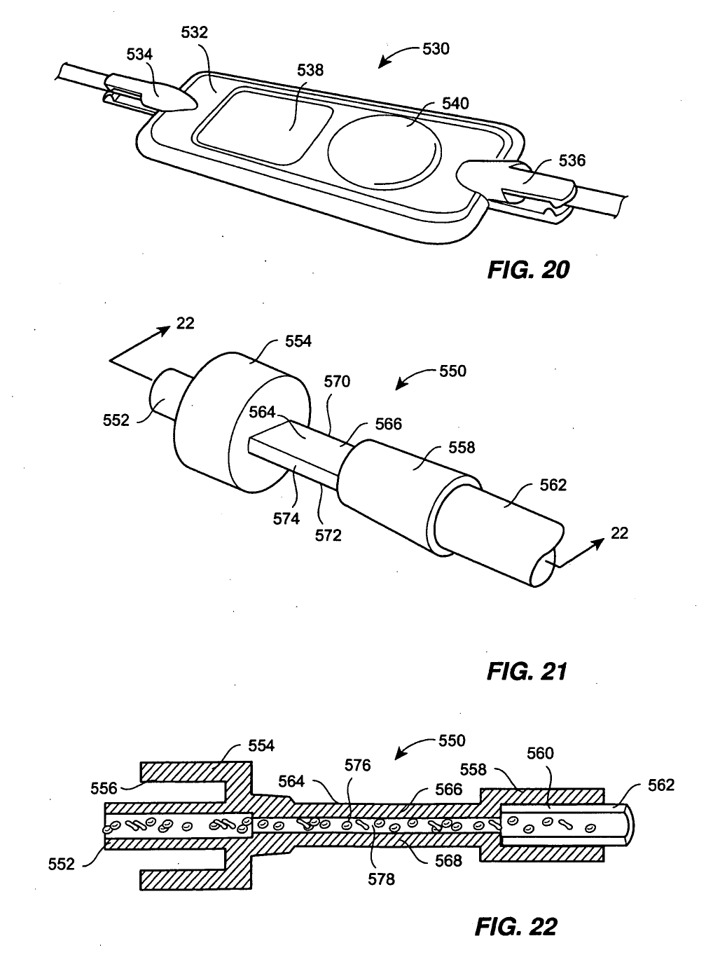

It will be recognized that the use of a one-way valve is not a requirement, but one or more on-off clamps may be used instead, as illustrated in FIG. 20. A device 530 includes a frame 532 to which two on-off clamps 534, 536, each of which may be moved between the “on” state and the “off” state manually, a sample cell 538 and a diaphragm 540 are attached. To operate the device 530, the medical practitioner closes the clamp 534 near the sample cell 538 after stopping the associated pump. Closure of the clamp 534 prevents fluid from passing through the inlet to the device 530. The practitioner compresses the diaphragm 540 to eject fluid out of the device 530. The practitioner then closes the clamp 536 near the diaphragm 540 and opens the clamp 534, after which the practitioner releases the diaphragm 540. This action facilitates suction of fluids from the body of the patient via catheter into the sample cell 538. This compression/release operation of the diaphragm 540 may be repeated until the sample cell 538 is filled with whole blood.

A further variant to the embodiment illustrated in FIG. 20 would feature clamps that are integrated with the diaphragm via a mechanical lever, such that one would performs the above-sequence of operations by pressing and releasing the diaphragm only. The lever would be engaged in the process of compressing the diaphragm, and the engagement of the lever would open and close the lever-controlled clamps. Such a system may be integrated with an analyzer (such as is illustrated in FIG. 14) to automate the entire process, although the analyzer may instead include particularly designed mechanism for opening/closing the clamps and compressing/releasing the diaphragm.

Having thus discussed the sensor cell in the context of an extension set wherein the sensor cell is defined by a one or more walls that are separate from the other structures of the extension set, such as the connectors, pump, valve, etc., FIGS. 21 and 22 illustrated an embodiment wherein the sensor cell is formed integrally with another structure of the extension set. In particular, the sensor cell is formed integrally with a male luer connector that defines, at least in part, the catheter hub connector. After this fashion, the sensor cell/connector may be used either with a separate infusion pump, such as in the example of FIGS. 10-16, or with a hand-operated flash bulb-type pump, such as in the example of FIG. 17.

In particular, an integrated sensor cell/connector 550 is illustrated in FIGS. 21 and 22. The connector 550 includes a male luer tip 552 surrounded by a collar 554 having a threaded surface 556. The male luer tip 552 and collar 554 thus define a luer lock. The connector 550 also includes a port 558 to receive an end 560 of tubing 562 of an extension set defined, at least in part, by the connector 550 and the tubing 562. To this extent, the connector 550 is shaped much like a convention luer lock connector of a conventional extension set.

It will be recognized that the connector 550 may be modified so that it could be used in addition to an extension set, for example between the extension set and the catheter hub or even between the extension set and the administration set. According to such a modification, the port 558 would be replaced with a female luer lock tip, instead of being sized to accept an end of a length of tubing. This would permit the sample cell/connector modification to be used in addition to other sets. For that matter, it will be recognized that the combination of the sample cell with one or more luer-type connectors does not preclude the combination of the sample cell with other forms of connectors.

Between the luer tip 552/collar 554 and the port 558 is a sample cell 564, defined by spaced walls 566, 568 bounded at opposing edges 570, 572 by end walls 574, 576. Similar to the sensor cell in FIG. 11, the cross-section of the sample cell 564, at least in the vicinity of a test region 578, is rectangular, while the cross-sections of passages in the luer tip 552 and the port 558 are circular or elliptical. While the transition between the passages of the luer tip 552 and the port 558 and the sample cell 564 is rather abrupt as illustrated, according to other examples a more gradual transition may be included instead.

As noted above, the sensor cell/connector 550 may be used either with an infusion pump or a hand-operated flash bulb to drawn blood into the sample cell 564. The placement of the sensor cell 564 so close to the catheter hub and associated catheter is advantageous in that it limits the distance that the blood must be drawn into the extension set to permit sensing and analysis to occur. To this extent, such a connector 550 may be particular well-suited for use with a hand-operated flash bulb. It will also be recognized that instead of integrating the sensor cell into the connector, the sensor cell may be integrated instead into the housing of the flash bulb instead.

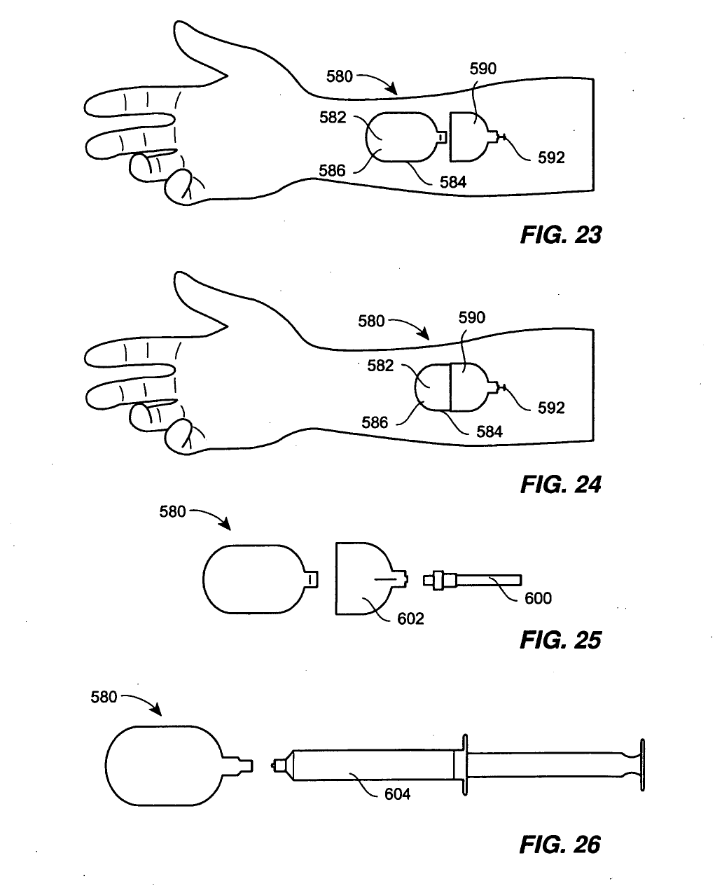

Still further variants as to the structure of the sensor cell are illustrated in FIGS. 23-26. According to these variants, the sensor cell and related inlet are defined by a structure that permits fluid to pass through the inlet, but provides no exit from the sensor cell. In this regard, the sensor cell operates similar to containers (or vacutainers) presently in use for drawing blood from an insertion site via a catheter hub or needle. However, the sensor cell is particular designed to be accommodated in an analyzer, such as is shown in FIGS. 13 and 14, for example.

Referring first to the example in FIG. 23, a sensor cell 580 is illustrated, which sensor cell 580 may have a structure similar to that illustrated in FIG. 11. In particular, the cell 580 includes a pair of spaced walls (one of which (582) is illustrated) that are joined at a peripheral edge 584 by a boundary wall 586. Unlike the example illustrated in FIG. 11, the boundary wall 586 has a single port that defines an inlet. The sample cell 580 may have no outlet, or may have a valve or other structure that allows air to exhaust from the cell 580 as the blood enters the cell 580. In another embodiment, the sample cell 580 may be pre-vacuumed.

The cell 580 may be used as is illustrated in FIGS. 23 and 24 in conjunction with an adapter, or access device, 590 associated with a catheter or needle 592 already inserted into the arm of a patient. While the catheter or needle 592 is illustrated inserted into a peripheral vein, the example is not so limited, but may be used with a central venous catheter for example. As illustrated, the sensor cell 580 may be initially separate (FIG. 23) and then attached to the adapter 590 (FIG. 24).

Once the cell 580 has been filled with blood, the cell 580 may be detached from the adapter 590 and inserted into the holder of an analyzer, such as the one illustrated in FIGS. 13 and 14. It will be recognized, however, that it would also be possible to design the cell 580, the adapter 590 and/or the holder of the analyzer so that the sample cell 580 could be disposed into the holder while still attached to the adapter.

Alternative methods may be used to fill the sensor cell 580. For example, as illustrated in FIG. 25, the sensor cell 580 may be used with an extension set 600 and an adapter 602 to permit the blood to be drawn at without detaching the extension set 600 from the catheter hub. FIG. 26 illustrates a still further alternative, wherein a syringe 604 is used to draw the blood from a catheter or other site, and then connected to the sensor cell 580 to fill the cell 580 with the drawn blood.

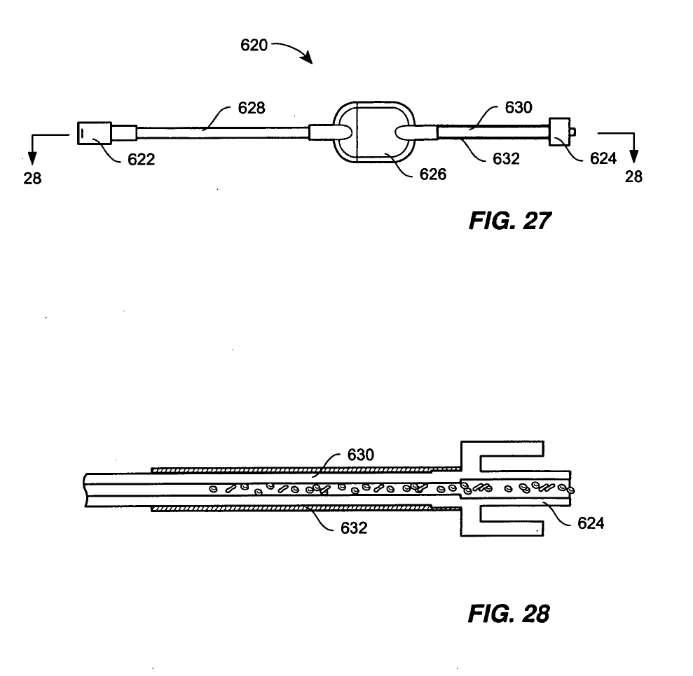

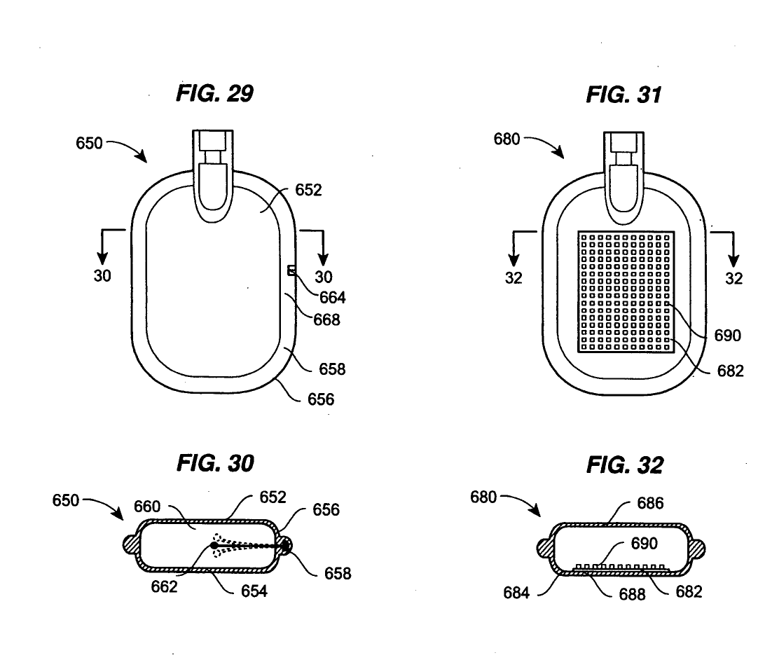

As will be recognized, many additional variants of the sensor cells and related assemblies illustrated above may be possible. FIGS. 27-32 illustrate further structures and/or features that may be combined with one or all of the embodiments illustrated above in FIGS. 10-26. That is, while the features illustrated in FIGS. 29-32 may be presented in the context of a particular example, such as the sensor cell of FIGS. 23-26, the disclosure is not so limited, and the features may in fact be used with the examples of FIGS. 10-22 as well. Further, while the features illustrated in FIGS. 27 and 28 are disclosed in the context of the examples of FIGS. 10-22, it will be recognized that the features may be used with the sensor cell of FIGS. 23-26 as well.

Referring first to FIGS. 27 and 28, an extension set 620 is illustrated, including an extension set connector 622, a catheter hub connector 624, a sensor cell 626 and tubing 628, 630 connecting the connectors 622, 624 to the sensor cell 626. In this regard, the example is similar to the extension set illustrated in FIGS. 11 and 12. However, as seen in FIG. 27, and to a greater degree in FIG. 28, the tubing 630 between the catheter hub connector 624 and the sensor cell 626, as well as the sensor cell 626 itself, may be covered with a layer 632 of material that has particular optical qualities.

Specifically, the material of the layer 632 transmits the wavelength utilized by the light emitter and the light receptor, but that does not transmit red. For example, the layer 632 may be defined by a translucent blue colored plastic layer. Such a layer 632 should absorb the red, so that fluid (e.g., blood) in the extension set 620 does not appear red. Where a blue layer is used, the fluid may appear blue or purple instead. In another embodiment, the layer 632 may be opaque.

The layer 632 may be disposed over the tubing 630 (and the cell 626) using a number of different techniques. For example, the layer 632 may be applied to the tubing 630 as a coating, by a spray device for example. Alternatively, the layer 632 may be formed separately from the tubing 630, and then disposed over the outside surface of the tubing 630, like a shield, sleeve or cover for example. The layer 632 may then be secured in place through the use of an adhesive according to certain examples. As a still further alternative, the layer 632 may be molded or co-extruded with the tubing 630.

The advantage in using such a material may be substantially psychological: while permitting the sensing and analysis to be conducted without hindrance, the material limits any discomfort the patient may experience in seeing blood pass up from the catheter into the extension set. Where the sensor cell is placed a distance from the catheter hub connector, this layer may be particularly helpful. However, even when the sample cell is integrated with the catheter hub connector, as in the example of FIGS. 21 and 22, applying the layer to the connector may be of some assistance in maintaining a reduced stress environment for the patient.

FIGS. 29 and 30 illustrate a sample cell 650 with opposing walls 652, 654 joined at a peripheral edge 656 by a boundary wall 658 to define a space 660. Disposed in the space 660 bounded by the walls 652, 654, 658 is a stirrer 662. As illustrated, the stirrer 662 is in the form of a cantilevered arm of piezoelectric material. The stirrer 662 may be controlled from outside the space 660 through a connection 664 provided in on an external surface 668 of the cell 650. However, it is recognized that other stirring devices may be disposed in the space 660, or the entire cell may be agitated or vibrated to cause a mixing of the material in the space 660 using acoustic waves or electrothermal, electrokinetic, magnetic or other mechanical stirring mechanisms, in the analyzer, for example. Stirring may increase mixing, and thus decrease reaction time. In another embodiment, microfeatures may be incorporated into the inner surfaces of the opposing walls 652, 654 to facilitate mixing.

In FIGS. 31 and 32, the sample cell 680 has an array 682 of materials disposed on an internal surface 684 of one of the walls 686, 688 that define the cell 680. Elements 690 of the array 682 may include materials that react or interact with the fluid or materials in the fluid in the cell 680, which reaction or interaction may cause the material to be bound to the array to simplify identification of the material. For example, the elements 690 of the array 682 may be biologics, such as ligands, antibodies, etc., that capture the biomarkers of interest (pathogens or other analytes). In fact, each element 690 of the array 682 may target a different bacterial strain, for example. As a consequence, more than one analyte may be diagnosed and/or quantitated in a single sample cell 680 by referencing which element 690 of the array 682 has bound with the captured bacteria. In another embodiment, arrays may be used to facilitate sensing based on chemical or biochemical reactions (e.g., a chemical or biochemical reaction occurs that provides an electrical or optical signal via an electrochemical or photochemical reaction).

As a still further alternative, particular useful for the cell 580 illustrated in FIGS. 23-26, for example, a membrane filter may be disposed at the inlet of the cell 580 (or the syringe 604, as in the example illustrated in FIG. 26). The filter may have a pore size of approximately at least 3-4 microns. Such a pore size is advantageous because most bacteria are smaller than 3-4 microns, while blood cells are usually larger, for example approximately 6-10 microns. Thus, the membrane filter will limit the passage of red and white blood cells into the cell 580 (or the syringe 604) while permitting plasma and pathogens, such as Staphylococcus Aureus, to pass into the cell 580. This may have the added benefit of limiting the fluorescence background signal generated by blood cells, thereby improving detection of the pathogens.

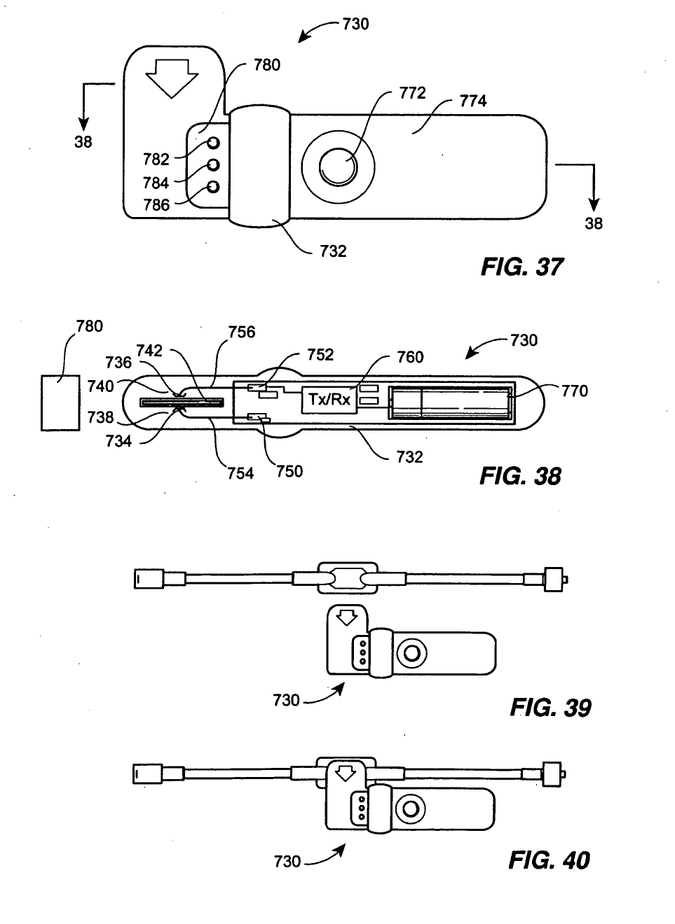

Having discussed a great number of different examples of sensor cell assemblies, it will also be recognized, as alluded to above, that the analyzer may also have more than one construction. FIGS. 33-40 illustrate peripherals that may be used in a version of the sensor system where certain interface features may be separated from the processing and signaling features of the system. In this regard, to the extent that it is necessary to provide power and/or light to the intravenous sensor, the peripheral device may include such interface capabilities while lacking other processing and signaling capabilities. As such, the peripherals may be operatively connected to the portions of the sensor system that include the processing and signaling features by a cable (FIGS. 33-36) or wirelessly (FIGS. 37-40). However, one advantage of separating certain of the interface features from the processing and signaling features is that the peripheral may remain attached to the sensor cell even when the sensor cell is not in use, given that the size of the housing may be reduced relative even to the hand-held unit illustrated in FIGS. 13 and 14.

Turning first to the example of a peripheral device 700 illustrated in FIGS. 33-36, the device 700 includes a housing 702, a light emitter 704 (including, for example, a colliminating lens) and a light receptor 706 (including, for example, a collection lens). The light emitter 704 and the light receptor 706 are disposed in the housing 702, with a first optical cable 708 connecting the light emitter 704 to a light source disposed outside the housing 702, and a second optical cable 710 connecting the light receptor 706 to a PMT, for example, also disposed outside the housing 702. As will be seen, the housing 702 has opposing walls 712, 714 that define a holder 716, with the light emitter 704 mounted to the wall 712 and the light receptor 706 mounted to the wall 714. The device 700 may also include a proximity switch 718 that is connected by a wire or line 720 to the processor, which switch 718 provides a signal indicative of the presence of a sensor cell within the holder 716 (compare FIGS. 35 and 36).

It will be recognized that the peripheral 700 permits the processor and signaling device (represented at 722 in FIG. 34, and including any of the elements of the analyzer illustrated in FIGS. 13 and 14 not present in the peripheral 700 illustrated in FIG. 34) to be disposed remotely to the extension set including the sensor cell, and thus remote to the patient. For purposes of this example, remote may refer to a distance of only several inches, such that the remainder of the analyzer is positioned in a bed with the patient, but not on the chest of the patient, for example. Alternatively, the cable 708, 710 and wire/line 720 may extend so that the remainder of the analyzer is positioned a bedside. While even greater distances may be possible, the wireless variant illustrated in FIGS. 37-40 may be better suited for such applications.

In use, the associated sample cell may be disposed in the peripheral 700 at all times, although the peripheral may only be attached during certain times of the day when sensing and analysis is performed. In fact, the peripheral 700 may be used in conjunction with a fully automated system that is coupled to an infusion system, like the system 210 illustrated in FIG. 10, either directly to the pump 220 or via the pump controller 222. As illustrated, the peripheral 700 does not provide an input that could be used to signal the remainder of the sensing system that the sensing and analysis process should be started, so the peripheral 700 is particular suited to a fully automated system. However, an input device could be added to the peripheral 700, if desired.

The sensor system may signal the pump 220 to stop operation, and to reverse the flow through the extension set so as to fill a sample cell. Once whole blood is detected in the sample cell, or after a certain time has elapsed, the sensor system may send a further signal to the pump 220 to stop operation. The sensor system may then perform the sensing step using the light emitter 704 and light receptor 706, and perform the analysis of the results. Based on the results, the system may signal the pump 220 to resume operation, may delay or terminate operation of the pump 220, may store the results of the analysis, and/or may cause a signaling device to actuate to alert a medical practitioner acting as caregiver to the patient.

As illustrated in FIGS. 37-40, a wireless peripheral device 730 may include a housing 732, a light emitter 734 and a light receptor 736. The light emitter 734 and the light receptor 736 may be disposed in the housing 732, as in the variant illustrated in FIGS. 33-36. Similarly, the housing 732 has opposing walls 738, 740 that define a holder 742, with the light emitter 734 mounted to the wall 738 and the light receptor 736 mounted to the wall 740. The sensor cell would be received within the holder 742 (compare FIGS. 39 and 40).

However, unlike the example illustrated in FIGS. 33-36, the device 730 includes a light source 750 and a PMT 752, with a first optical cable 754 connecting the light emitter 734 to the light source 750 and a second optical cable 756 connecting the light receptor 736 to the PMT 752. As a consequence, the device 730 is capable of producing an electrical signal (e.g., a voltage signal). Moreover, the electrical signal may be provided to an on-board a wireless transmitter 760 that is in wireless communication with a wireless receiver coupled to the processing/signaling unit (represented at 780 in FIG. 38). It will be recognized that the transmitter 760 may take the form of a transceiver, as illustrated, although the capability of the device 730 to receive as well as transmit signals is not a requirement for all examples. For that matter, a separate wireless receiver could be provided instead. The transmitter 760 may operate on radio frequency wavelengths, or in the infrared portion of the spectrum, for example. Furthermore, in another embodiment, process/analysis features may be included in the device 730, the device 730 transmitting the processed data/result to a medical practitioner or medical information system (represented in this case by 780 in FIG. 38).

The device 730 may also include an on-board power supply 770 that may be rechargeable or disposable. The power supply 770 may be active continuously, or the power supply may provide power to the components of the device 730 only when the peripheral 730 is to be used to sense a patient's condition, as reflected by a caregiver or medical practitioner depressing a button 772 disposed on an exterior surface 774 of the device 730.

In use, the associated sample cell may be disposed in the peripheral 730 at all times, although the peripheral may only be attached during certain times of the day when sensing and analysis is performed. In fact, the peripheral 730 may be used in conjunction with a fully automated system that is coupled to an infusion system, like the system 210 illustrated in FIG. 10, either directly to the pump 220 or via the pump controller 222. As illustrated, the peripheral 730 provides an input, in the form of the button 772, that could be used to signal the remainder of the sensing system that the sensing and analysis process should be started, so the peripheral 730 is particular suited to such a system. However, the peripheral 730 could be modified to remove the button 772, if a fully automated system is desired.

The sensor system may signal the pump 220 to stop operation, and to reverse the flow through the extension set so as to fill a sample cell. Once whole blood is detected in the sample cell, or after a certain time has elapsed, the sensor system may send a further signal to the pump 220 to stop operation. The sensor system may then perform the sensing step using the light emitter 704 and light receptor 706, and perform the analysis of the results. Based on the results, the system may signal the pump 220 to resume operation, may delay or terminate operation of the pump 220, may store the results of the analysis, and/or may cause a signaling device to actuate to alert a medical practitioner acting as caregiver to the patient.

For example, the peripheral 730 includes a signaling device 780, including one or more light elements 782, 784, 786. A signal received by the transceiver 760 may be passed to the signaling device to provide a suitable visual indication to the caregiver. For example, the light elements 782, 784, 786 may be light emitting diodes (LEDs) with or without associated colored covers, such that the light element 782 gives off a red light, the light element 784 a yellow light and the light element 786 a green light. As a consequence, the caregiver could be apprised by red, yellow or green light that the patient is either in a fully compromised, partially compromised or healthy condition. In the alternative or in addition, an aural indication may be provided, via a buzzer or other sound device.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

We claim:

1. An integrated sensor system for providing information to a control system, comprising:
a catheter configured for communication with the control system, the catheter forming at least one lumen; and
at least one sensor disposed within the catheter, the at least one sensor comprising at least one of an optical sensor, an electrical sensor or a chemical/biochemical sensor.

2. The integrated sensor system of claim 1, where in the catheter further comprises side ports to enable blood flow into the at least one lumen to perform sensing

3. The integrated sensor system of claim 1, wherein the at least one sensor comprises an optical sensor having at least one optical fiber for emitting light and at least one optical fiber for collecting light.

4. The integrated sensor system of claim 1, wherein the at least one sensor comprises an electrical sensor having an anode, a cathode and a reference electrode.

5. The integrated sensor system of claim 1, wherein the at least one sensor comprises a chemical/biochemical sensor generating an optical or electrical signal according to a chemical or biochemical reaction.

6. An integrated sensor system, comprising:
an infusion pump;
a control system operably connected to the infusion pump;
a multi-lumen catheter in fluid communication with the infusion pump; and
at least one sensor disposed within the catheter, the at least one sensor comprising at least one of an optical sensor, an electrical sensor or a chemical/biochemical sensor, the sensor operably connected to the control system,
wherein the control system is configured to receive and process input signals from the at least one sensor and to provide an output useful for a real-time diagnosis.

7. A sensor system comprising:
a sample cell including opposing walls spaced from each other to define a test region therebetween and an inlet in fluid communication with the test region; and
an analyzer including:
a housing comprising a holder in which at least the test region of the sample cell is received,
a light emitter and a light receptor, the light emitter and the light receptor disposed about the holder adjacent to the test region;
a processor operatively coupled to the light receptor to receive a sensor signal therefrom, the processor programmed to determine a physical condition of a patient according to the sensor signal; and
signaling device operatively coupled to the processor to receive a processor signal therefrom, the signaling device provides an indication associated with the physical condition of the patient according to the processor signal.

8. The sensor system of claim 7, further comprising an extension set, the extension set having an administration set connector and a catheter hub connector, the sample cell formed with the extension set between the administration set connector and the catheter hub connector.

9. The sensor system of claim 8, further comprising an administration set coupled to the extension set and a reversible pump operatively coupled to the administration set, the pump having a forward state to pass fluid through the extension set from the administration set connector to the catheter hub connector and a reverse state to pass fluid through the extension set from the catheter hub connector to the administration set connector.

10. The sensor system of claim 8, wherein the extension set comprises a flexible diaphragm disposed between the administration set connector and the sample cell, the diaphragm moveable between a depressed state and a distended state to draw fluid into the sample cell.

11. The sensor system of claim 10, wherein the extension set comprises at least one on-off clamp, the on-off clamp open to permit fluid to flow in the direction from the administration set connector to the catheter hub connector and closed to limit flow in the direction from the catheter hub connector to the administration set connector.

12. The sensor system of claim 11, further comprising a frame, the sample cell, the diaphragm, and the at least one on-off clamp being attached to the frame, the frame having a first port coupled to the extension set connector and a second port coupled to the catheter hub connector.

13. The sensor system of claim 7, wherein the at least one of the opposing walls is defined in whole or in part by quartz or ultraviolet-grade fused silica.

14. The sensor system of claim 7, wherein the sample cell is open only at the inlet, and the inlet is attached to a catheter hub connector.

15. The sensor system of claim 7, wherein the light emitter and the light receptor are disposed in the housing to define a peripheral device, the peripheral device being detached from the processor.

16. The sensor system of claim 15, wherein the peripheral device is operatively coupled to the processor by a length of cable.

17. The sensor system of claim 15, wherein the peripheral device comprises a wireless transmitter coupled to the light receptor and the processor has a wireless receiver coupled thereto and in wireless communication with the wireless transmitter.

18. A sensor system disposable including:
an administration set connector;
a catheter hub connector; and
a sensor cell including opposing walls spaced from each other to define a test region therebetween, the sample cell connected at a first end to the administration set connector and at a second end to the catheter hub connector.

19. The sensor system disposable of claim 18, further comprising a flexible diaphragm disposed between the administration set connector and the sample cell, the diaphragm moveable between a depressed state and a distended state to draw fluid into the sample cell.

20. The sensor system disposable of claim 19, wherein the extension set comprises at least one on-off clamp, the at least one on-off clamp open to permit fluid to flow in the direction from the administration set connector to the catheter hub connector and closed to limit flow in the direction from the catheter hub connector to the administration set connector.

21. The sensor system disposable of claim 20, further comprising a frame, the sample cell, the diaphragm, and the at least one on-off clamp being attached to the frame, the frame having a first port coupled to the extension set connector and a second port coupled to the catheter hub connector.
